SEQUENCE LISTING (12 sequences)

SEQ ID NO: 1 - DNA, 3088 bp, human connexin 43
acaaaaaagcttttacgaggtatcagcacttttctttcattagggggaaggcgtgaggaaagtaccaaacagcagcggagttttaaactttaaatagacaggtctgagtgcctgaacttgccttttcattttacttcatcctccaaggagttcaatcacttggcgtgacttcactacttttaagcaaaagagtggtgcccaggcaacatgggtgactggagcgccttaggcaaactccttgacaaggttcaagcctactcaactgctggagggaaggtgtggctgtcagtacttttcattttccgaatcctgctgctggggacagcggttgagtcagcctggggagatgagcagtctgcctttcgttgtaacactcagcaacctggttgtgaaaatgtctgctatgacaagtctttcccaatctctcatgtgcgcttctgggtcctgcagatcatatttgtgtctgtacccacactcttgtacctggctcatgtgttctatgtgatgcgaaaggaagagaaactgaacaagaaagaggaagaactcaaggttgcccaaactgatggtgtcaatgtggacatgcacttgaagcagattgagataaagaagttcaagtacggtattgaagagcatggtaaggtgaaaatgcgaggggggttgctgcgaacctacatcatcagtatcctcttcaagtctatctttgaggtggccttcttgctgatccagtggtacatctatggattcagcttgagtgctgtttacacttgcaaaagagatccctgcccacatcaggtggactgtttcctctctcgccccacggagaaaaccatcttcatcatcttcatgctggtggtgtccttggtgtccctggccttgaatatcattgaactcttctatgttttcttcaagggcgttaaggatcgggttaagggaaagagcgacccttaccatgcgaccagtggtgcgctgagccctgccaaagactgtgggtctcaaaaatatgcttatttcaatggctgctcctcaccaaccgctcccctctcgcctatgtctcctcctgggtacaagctggttactggcgacagaaacaattcttcttgccgcaattacaacaagcaagcaagtgagcaaaactgggctaattacagtgcagaacaaaatcgaatggggcaggcgggaagcaccatctctaactcccatgcacagccttttgatttccccgatgataaccagaattctaaaaaactagctgctggacatgaattacagccactagccattgtggaccagcgaccttcaagcagagccagcagtcgtgccagcagcagacctcggcctgatgacctggagatctagatacaggcttgaaagcatcaagattccactcaattgtggagaagaaaaaaggtgctgtagaaagtgcaccaggtgttaattttgatccggtggaggtggtactcaacagccttattcatgaggcttagaaaacacaaagacattagaatacctaggttcactgggggtgtatggggtagatgggtggagagggaggggataagagaggtgcatgttggtatttaaagtagtggattcaaagaacttagattataaataagagttccattaggtgatacatagataagggctttttctccccgcaaacacccctaagaatggttctgtgtatgtgaatgagcgggtggtaattgtggctaaatatttttgttttaccaagaaactgaaataattctggccaggaataaatacttcctgaacatcttaggtcttttcaacaagaaaaagacagaggattgtccttaagtccctgctaaaacattccattgttaaaatttgcactttgaaggtaagctttctaggcctgaccctccaggtgtcaatggacttgtgctactatatttttttattcttggtatcagtttaaaattcagacaaggcccacagaataagattttccatgcatttgcaaatacgtatattctttttccatccacttgcacaatatcattaccatcactttttcatcattcctcagctactactcacattcatttaatggtttctgtaaacatttttaagacagttgggatgtcacttaacattttttttttttgagctaaagtcagggaatcaagccatgcttaatatttaacaatcacttatatgtgtgtcgaagagtttgttttgtttgtcatgtattggtacaagcagatacagtataaactcacaaacacagatttgaaaataatgcacatatggtgttcaaatttgaacctttctcatggatttttgtggtgtgggccaatatggtgtttacattatataattcctgctgtggcaagtaaagcacactttttttttctcctaaaatgtttttccctgtgtatcctattatggatactggttttgttaattatgattctttattttctctcctttttttaggatatagcagtaatgctattactgaaatgaatttcctttttctgaaatgtaatcattgatgcttgaatgatagaattttagtactgtaaacaggctttagtcattaatgtgagagacttagaaaaaatgcttagagtggactattaaatgtgcctaaatgaattttgcagtaactggtattcttgggttttcctacttaatacacagtaattcagaacttgtattctattatgagtttagcagtcttttggagtgaccagcaactttgatgtttgcactaagattttatttggaatgcaagagaggttgaaagaggattcagtagtacacatacaactaatttatttgaactatatgttgaagacatctaccagtttctccaaatgccttttttaaaactcatcacagaagattggtgaaaatgctgagtatgacacttttcttcttgcatgcatgtcagctacataaacagttttgtacaatgaaaattactaatttgtttgacattccatgttaaactacggtcatgttcagcttcattgcatgtaatgtagacctagtccatcagatcatgtgttctggagagtgttctttattcaataaagttttaatttagtataaacat

SEQ ID NO: 2 - DNA, 3148 bp, rat connexin 43
acgaggtatcagcacttttctttcattgggggaaaggcgtgaggaaagtaccaaacagcagcagacttttaaactttaaacagacaggtctgagagcctgaactctcatttttcctttgacttcagcctccaaggagttccaccaactttggcgcgccggcttcactttcattaagtgaaagagaggtgcccagacatgggtgactggagtgccttggggaagcttctggacaaggtccaagcctactccaccgctggagggaaggtgtggctgtcagtgctcttcatattcagaatcctgctcctggggacagctgttgagtcagcttggggtgatgaacagtctgcctttcgctgtaacactcaacaacctggctgcgaaaacgtctgctatgacaagtccttccccatctctcacgtgcgcttctgggtccttcagatcatattcgtgtctgtgcccaccctcctgtacttggcccatgtgttctatgtgatgaggaaggaagagaagctaaacaagaaagaagaggagctcaaagtggcccagactgacggggtcaacgtggagatgcacctgaagcagattgaaatcaagaagttcaagtacgggattgaagagcacggcaaggtgaaaatgaggggcggcttgctgagaacctacatcatcagcatcctcttcaagtctgtcttcgaggtggccttcctgctcatccagtggtacatctatgggttcagcttgagcgcggtctacacctgcaagagagatccctgcccccaccaggtagactgcttcctctcacgtcccacggagaaaaccatcttcatcatcttcatgctggtggtgtccttggtgtctctcgctttgaacatcattgagctcttctacgtcttcttcaaaggcgttaaggatcgcgtgaagggaagaagcgatccttaccacgccaccactggcccactgagcccatcaaaagactgcggatctccaaaatacgcctacttcaatggctgctcctcaccaacggctccactctcgcctatgtctcctcctgggtacaagctggttactggtgacagaaacaattcctcgtgccgcaattacaacaagcaagctagcgagcaaaactgggcgaactacagcgcagagcaaaatcgcatggggcaggccggaagcaccatctccaactcgcacgcccagccgttcgatttccccgacgacaaccagaatgccaaaaaagttgctgctggacatgaactccagccattagccatcgtggaccaacgaccttccagcagagccagcagccgcgccagcagcaggcctcggcctgatgacctggagatttaaaccggcttgagcatcgagctgtcgattatggaggagaaaaaaaggtgcttgcagaacatgcacctagggtgttcattttcgttcccgtggagttggtactcaacaacctcagcaatgaggcatagaaaacaaagacattacaatacctaggttccttgggggtgcttgggatagctgggcggaacaggtggggataagggaggtacacattgatatttaatgtagaagattcaaagaacttaaattctagtaagagtcgcattgggtgaacatagataagggctttctctctctgcccccatcccccaactgaaccttaagaatgagtctgtgtacatgaattgagtgagtgatatttttttttaattttgttttactgagattctgcgatagctttgagcagaaatctgagtcctccacatagcgtttcctttatgaaaagacagattgtcctacatcccccccccccaaaaaaaacattccagtgtttaaaaacttgcattttgcaggtaagcttccctggtctacccctctgggtgtgagtggaccttgtgttatttgctagacacgattttcattcttagtatcaatctgaagttcagacaaggctcaaagaaaaacgaccgcccatgtagttgcatctcactgggttctttgctgaatctgtctcacctttgtgccttccaaacatcatcctcgactggtcctcgccctcaccaaacggcttctatcaacatttttaaaacagtcataacgtcatttttagtttagttttttttttttcttttgagttagcatcagggaggtgagccatgctcaagatttaacaatcgcttctacgtgtggatgtgcgcatgtgtaagtgtgtgttttgtcattattggtacaagcagaggcagtctagactcacaaatacagatttgaatctaatgcacagtgttcaaatttgaaccttcctagtggatttttgtggtgtgggccaatgcggtgtttacattatagaattcctgccgtgcaagtaaagcacagtttttccctgaaaattttttttccaggtattccattatggatactggttttgttaattataatttctctctctctctctctctctctctctctctctctctctctctctctcttttagaatgtagcagtaatcgccattactgaaatgaatgattttctttttttttgaaatataatcattgatgcttgaatgatagagttttagtactgtaaacgggctttagtcattaatgtgagagacttagatggggattgcctagagtggactatcaagtgagcctaaaggaactttgtagtaactggtaatctgattttttttttgtcctacttaactacacataactcagaacttgtattctgagtttaacagtcttttagattgacgagcaacttggatgtttgcactaagactttgtttgaaacactaaggggcgagggaggtttcaacagtgcacatgtaactaatttatttgaactgtatgctaaagacaccagtttcttcaaatgactttttttaaaaaaaaacaaaaacaaaactcatcacagatgattgaaatgtcgagttatcatgtgtttccctcttgcgcgtcagctacagaaaagagtttttgtacaatgagaaactaatgtgtttgacattccacgttaaactgctgtcgtgttcggcttcgtagcatgtaatgtagacctagcccatccattcatgtgctcgggagagtgttctttatccaataaagttttaatttagtatataataaaaaaaaaaaaaaaaaa

SEQ ID NO: 3 - DNA, 20 bp, human
caattcttcttgccgcaatt

SEQ ID NO: 4 - DNA, 20 bp, rat
caattcctcgtgccgcaatt

SEQ ID NO: 5 - DNA, 20 bp, rat connexin 43
caauuccucgugccgcaatt

SEQ ID NO: 6 - DNA, 20 bp, rat connexin 43
uugcggcacgaggaauugtt

SEQ ID NO: 7 - DNA, 20 bp, human connexin 43
caauucuucuugccgcaatt

SEQ ID NO: 8 - DNA, 20 bp, human connexin 43
uugcggcaagaagaauugtt

SEQ ID NO: 9 - DNA, 30 bp, rat connexin 43
  antisense origodeoxyribonucleotide
gtaattgcggcaggaggaattgtttctgtc

SEQ ID NO: 10 - DNA, 30 bp, rat connexin 43
  sense origodeoxyribonucleotide
gacagaaacaattcctcctgccgcaattac

SEQ ID NO: 11 - DNA, 21 bp, Artificial sequence
  nonfunctional control siRNA
aauucuccgaacgugucacgt

SEQ ID NO: 12 - DNA, 21 bp, Artificial sequence
  nonfunctional control siRNA
gugacacguucggagaauutt

(19) United States
(12) Patent Application Publication
(10) Pub. No.: US 2009/0163432 A1
(43) Pub. Date: Jun. 25, 2009

(54) Therapeutic Agent for Corneal Diseases

(75) Inventors: Tetsuro Takamatsu, Kyoto (JP); Ping Dai, Kyoto (JP); Shigeru Kinoshita, Kyoto (JP)

Correspondence Address: KRATZ, QUINTOS & HANSON, LLP, 1420 K Street, N.W., Suite 400, WASHINGTON, DC, 20005, US

(73) Assignee: Kansai Technology Licensing Organization Co., Ltd., Kyoto-shi (JP)

(21) Appl. No.: 12/084,645

(22) PCT Filed: Nov. 8, 2006
(86) PCT No.: PCT/JP2006/322239

§ 371 Date: May 7, 2008

(30) Foreign Application Priority Data
Nov. 8, 2005 (JP) 2005-323844

(51) Int. Cl. A61K 31/7105; C07H 21/02

(52) U.S. Cl. 514/44; 536/24.5

(57) ABSTRACT

The present invention relates to a treatment agent for a disease or a disorder caused by a reduction in corneal endothelial cells, comprising as an active component at least one nucleic acid molecule inhibiting the expression of a connexin 43 gene.

### TECHNICAL FIELD

The present invention relates to the proliferation of corneal endothelial cells or the regeneration of corneal endothelium, and particularly relates to a treatment agent for treating a disease or a disorder caused by a reduction in corneal endothelial cells, the use thereof for such treatment, a pharmaceutical composition for such treatment, a method for treating the disease or the disorder, a treatment agent and a treatment method allowing an intraocular surgery in a patient having reduced corneal endothelial cells, and siRNA for human connexin 43 (Cx43).

### BACKGROUND ART

The cornea is a transparent tissue having the role of an optical lens, and has corneal endothelial tissue as its innermost layer.

Corneal endothelial cells are indispensable for maintaining the transparency of the cornea. When a human corneal endothelial cell is once impaired, it is not regenerated and a impaired portion is covered by the migration of the remaining surrounding endothelial cells. However, when damage above a certain level is inflicted upon the corneal endothelium to thereby reduce the thickness of the corneal endothelium, the transparency of the cornea cannot be maintained resulting in bullous keratopathy. A cornea transplant is required to treat this disease.

Qiu et al has reported in Non-patent Literature 1 that an antisense oligonucleotide (AS ODN) sequence of connexin 43 (Cx43), composed of 30 particular nucleotides is effective in the recovery of skin wounds.

Non-patent Literature 2 has reported that Cx43 has an effect on inhibition of tumors.

Patent Document 1 has disclosed various RNAi of connexin, but has not suggested their association with the corneal endothelial cell.

Patent Document 2 has disclosed that the inhibition of connexin protein expression by an antisense polynucleotide is effective for the reduction of neuron cell death, wound healing, reduction of inflammation, reduction of scarring, and rejuvenation and thickening of the skin.

Non-patent Literature 1: Qiu et al., “Targeting connexin expression accelerates the rate of wound repair”, Current Biology 13, 1697-1703, 2003

Non-patent Literature 2: You-Wei Zhang et al., J. Biol. Chem., Vol. 278 No. 45, 44852-44856, 2003

Patent Document 1: US2005/0119211

Patent Document 2: WO00/44409

### DISCLOSURE OF INVENTION

### Problem to be Solved by the Invention

It is an object of the present invention to provide a technology to proliferate or regenerate a corneal endothelial cell.

### Means for Solving the Problem

As a result of extensive study in light of the above problems, the present inventors found that corneal endothelial cells are caused to proliferate by inhibiting the expression of a Cx43 gene, and completed the present invention.

The present invention provides the following items [1] to [31].

[1] A treatment agent for a disease or a disorder caused by a reduction in corneal endothelial cells, comprising as an active component at least one nucleic acid molecule inhibiting the expression of a connexin 43 gene.

[2] The treatment agent according to [1] wherein the disease or the disorder caused by a reduction in the corneal endothelial cells is bullous keratopathy.

[3] The treatment agent according to [1] which is in a form of a formulation for administering to an anterior chamber of an eye.

[4] The treatment agent according to [1] wherein the nucleic acid molecule is siRNA comprising a polynucleotide sequence of 15 to 30 consecutive nucleotides having a sequence complementary to the connexin 43 gene.

[5] The treatment agent according to [1] wherein the nucleic acid molecule is siRNA having the following sequence:

| | |
|---|---|
| Sense: | 5′-CAA UUC UUC UUG CCG CAA TT-3′ |
| Antisense: | 5′-UUG CGG CAA GAA GAA UUG TT-3′ |

for human connexin 43.

[6] Use of at least one nucleic acid molecule inhibiting the expression of a connexin 43 gene, for treating a disease or a disorder caused by a reduction in corneal endothelial cells.

[7] The use according to [6] wherein the disease or the disorder caused by a reduction in corneal endothelial cells is bullous keratopathy.

[8] The use according to [6] for administering the nucleic acid molecule to an anterior chamber of an eye.

[9] The use according to [6] wherein the nucleic acid molecule is siRNA comprising a polynucleotide sequence of 15 to 30 consecutive nucleotides having a sequence complementary to the connexin 43 gene.

[10] The use according to [6] wherein the nucleic acid molecule is siRNA having the following sequence:

| | |
|---|---|
| Sense: | 5′-CAA UUC UUC UUG CCG CAA TT-3′ |
| Antisense: | 5′-UUG CGG CAA GAA GAA UUG TT-3′ |

for human connexin 43.

[11] A method for treating a disease or a disorder caused by a reduction in corneal endothelial cells, wherein at least one nucleic acid molecule inhibiting the expression of a connexin 43 gene is administered to a patient having the disease or the disorder caused by the reduction in corneal endothelial cells.

[12] The method according to [11] wherein the disease or the disorder caused by the reduction in corneal endothelial cells is bullous keratopathy.

[13] The method according to [11] wherein the nucleic acid molecule is administered to an anterior chamber of an eye.

[14] The method according to [11] wherein the nucleic acid molecule is siRNA comprising a polynucleotide sequence of 15 to 30 consecutive nucleotides having a sequence complementary to the connexin 43 gene.

[15] The method according to [11] wherein the nucleic acid molecule is siRNA having the following sequence:

| | |
|---|---|
| Sense: | 5′-CAA UUC UUC UUG CCG CAA TT-3′ |
| Antisense: | 5′-UUG CGG CAA GAA GAA UUG TT-3′ |

for human connexin 43.

[16] A pharmaceutical composition for treating a disease or a disorder caused by a reduction in corneal endothelial cells, comprising an effective amount of at least one nucleic acid molecule inhibiting the expression of a connexin 43 gene, and a pharmaceutically acceptable carrier, excipient or diluent.

[17] The pharmaceutical composition according to [16] wherein the disease or the disorder caused by the reduction in corneal endothelial cells is bullous keratopathy.

[18] The pharmaceutical composition according to [16] which is in a form of a formulation for administering to an anterior chamber of an eye.

[19] The pharmaceutical composition according to [16] wherein the nucleic acid molecule is siRNA comprising a polynucleotide sequence of 15 to 30 consecutive nucleotides having a sequence complementary to the connexin 43 gene.

[20] The pharmaceutical composition according to [16] wherein the nucleic acid molecule is siRNA having the following sequence:

| | |
|---|---|
| Sense: | 5′-CAA UUC UUC UUG CCG CAA TT-3′ |
| Antisense: | 5′-UUG CGG CAA GAA GAA UUG TT-3′ |

for human connexin 43.

[21] A treatment agent for allowing an intraocular surgery in a patient who cannot undergo the intraocular surgery due to a reduction in corneal endothelial cells, comprising as an active component at least one nucleic acid molecule inhibiting the expression of a connexin 43 gene.

[22] The treatment agent according to [21] wherein the patient is a patient suspected of having bullous keratopathy due to an intraocular surgery.

[23] The treatment agent according to [21] which is in a form of a formulation for administering to an anterior chamber of an eye.

[24] The treatment agent according to [21] wherein the nucleic acid molecule is siRNA comprising a polynucleotide sequence of 15 to 30 consecutive nucleotides having a sequence complementary to a connexin 43 gene.

[25] The treatment agent according to [21] wherein the nucleic acid molecule is siRNA having the following sequence:

| | |
|---|---|
| sense: | 5′-CAA UUC UUC UUG CCG CAA TT-3′ |
| antisense: | 5′-UUG CGG CAA GAA GAA UUG TT-3′ |

for human connexin 43.

[26] A treatment method for allowing an intraocular surgery wherein at least one nucleic acid molecule inhibiting the expression of a connexin 43 gene is administered before the intraocular surgery in a patient where corneal endothelial cells are reduced, where the intraocular surgery is required but a disease or a disorder caused by a reduction in corneal endothelial cells is likely to occur due to the surgery.

[27] The method according to [26] wherein the patient is likely to develop bullous keratopathy due to the intraocular surgery.

[28] The method according to [26] wherein the nucleic acid molecule is administered to an anterior chamber of an eye.

[29] The method according to [26] wherein the nucleic acid molecule is siRNA comprising a polynucleotide sequence of 15 to 30 consecutive nucleotides having a sequence complementary to the connexin 43 gene.

[30] The method according to [26] wherein the nucleic acid molecule is siRNA having the following sequence:

| | |
|---|---|
| Sense: | 5′-CAA UUC UUC UUG CCG CAA TT-3′ |
| Antisense: | 5′-UUG CGG CAA GAA GAA UUG TT-3′ |

for human connexin 43.

[31] siRNA having the following sequence:

| | |
|---|---|
| sense: | 5′-CAA UUC UUC UUG CCG CAA TT-3′ |
| antisense: | 5′-UUG CGG CAA GAA GAA UUG TT-3′ |

for human connexin 43.

### EFFECTS OF THE INVENTION

Corneal endothelial cells are a monolayer cell layer present on the rear side of the cornea, and play an important role in maintaining transparency by maintaining a constant water content in the cornea with a pump function and a barrier function. Once the corneal endothelial cells are impaired and drop off due to an invasive intrusion, such as those occurring in external injury, dystrophy or intraocular surgery, corneal endothelium dysfunction occurs causing strong edema and opacity in the cornea because corneal endothelial cells in primates such as human beings and monkeys have little ability to proliferate in vivo. Such a pathological state is referred to as bullous keratopathy, where the patient develops a severe visual impairment. A full thickness cornea transplant is currently performed for advanced bullous keratopathy, but its long term results are worse than those for other corneal diseases, and the development of better therapeutic methods is desired. Also in eye banks in Japan, the donated corneas required for such cornea transplants are always in short supply, and patients with bullous keratopathy requiring such a transplant are currently forced to wait for a long time. Under such a social and medical context, the present inventors developed a new method for treating corneal endothelial diseases.

According to the present invention, it is possible to proliferate the corneal endothelial cells and effectively treat a disease or a disorder caused by a reduction in corneal endothelial cells.

For example, corneal endothelial cells can be proliferated to restore or regenerate the corneal endothelial tissue leading to the healing of bullous keratopathy by administering at least one nucleic acid molecule inhibiting the expression of a connexin 43 gene to an anterior chamber of an eye in a patient with bullous keratopathy whose cornea has low transparency.

Even when the corneal endothelial tissue becomes thin due to various causes, e.g., an external injury to the eye, an intraocular infection, an increase of intraocular pressure due to glaucoma or insufficient oxygen due to contact lenses, and thus surgery requiring an intraocular manipulation (intraocular surgery) or laser therapy cannot be given, the intraocular surgery can be allowed by administering at least one nucleic acid molecule inhibiting the expression of the connexin 43 gene to cause the corneal endothelial cells to proliferate and thicken the corneal endothelial tissue.

### MODES FOR CARRYING OUT THE INVENTION

Intraocular surgery herein includes surgery requiring intraocular manipulation and laser therapy. Such intraocular surgery includes, but is not limited to, surgeries for cataract, glaucoma and strabismus, retinal detachment operations and vitreous surgery.

In the present invention, the “disease or disorder due to a reduction in corneal endothelial cells” includes not only bullous keratopathy but also states where because of reduced corneal endothelial cells, the risk of developing bullous keratopathy due to intraocular surgery or laser therapy is increased and such intraocular surgery or laser therapy cannot be performed.

The cause of bullous keratopathy mainly includes intraocular surgery, and further includes genetic diseases of the corneal endothelium (reduction in corneal endothelial cells with aging), corneal endotheliitis due to infection with the herpes virus, external injury and dystrophy.

Connexin 43 (Cx43) is known to be involved in intercellular communication through gap junctions and channel formation in cardiac muscle tissues. The gene sequences and amino acid sequences of connexin 43 in human beings, monkeys and rats are known publicly, as shown in the following Table 1.

TABLE 1

| | Accession Nos. of database of |
|---|---|
| Origin of Cx43 | gene or amino acid sequences |
| Human beings (SEQ ID: No. 1) | NM_000165 |
| Rats (SEQ ID: No. 2) | NM_012567 |
| Monkeys | AB169817 |

The Cx43 whose gene expression is inhibited in the present invention is a protein that constitutes a gap junction. A human Cx43 gene sequence and a rat Cx43 gene sequence are described in SEQ ID NOS:1 and 2, respectively, and a preferable target gene is human Cx43.

Connexin molecules are classified as Cx43, Cx26 and Cx32 by molecular weight, and the protein involved in the proliferation of corneal endothelial cells is Cx43. It is also possible to simultaneously inhibit the gene expression of Cx26 and Cx32 in addition to Cx43, but in preferable embodiments, Cx43 is specifically inhibited.

Qiu et al. (Current Biology 13, 1697-1703, 2003) has described that the knockdown of Cx43 using an antisense oligodeoxynucleotide decreases the number of neutrophils to thereby reduce inflammation and promote wound healing, but such inflammation scarcely occurs in the cornea, and thus the inhibition of such inflammation scarcely affects the cornea. Corneal endothelial cells have been recognized as cells that do not proliferate before the application of the present invention. The present inventors have found, surprisingly, that the proliferation of corneal endothelial cells can be promoted by inhibiting Cx43.

The nucleic acid molecule of the present invention includes the antisense DNA, antisense RNA and siRNA (small interfering RNA; RNAi) of connexin 43, and siRNA is preferably exemplified. siRNA has a polynucleotide sequence of 15 to 30 consecutive complementary nucleotides, preferably 18 to 25 nucleotides and more preferably 19 to 21 nucleotides. siRNA may be used alone or in a combination of two or more molecules. In siRNA, a complementary chain region may have 15 to 30, preferably 18 to 25 and more preferably 19 to 21 nucleotides. Also, siRNA may be composed of two complementary strands of RNA, and may take a structure in which one or both sides of these two RNA strands are ligated to a nucleic acid sequence of an appropriate length. In the latter case, siRNA becomes a single strand or cyclic RNA having a complementary region and a non-complementary region.

The nucleic acid molecule of the present invention may be made by making each complementary chain independently and then joining them, or it may be made as a single strand. The nucleic acid molecule may be synthesized chemically or made using a recombinant gene technology. Specifically, the nucleic acid molecule is preferably synthesized chemically using a protected ribonucleotide phosphoramidite method and a suitable DNA/RNA synthesizer. The polynucleotide may be synthesized in person using a commercially available DNA/RNA synthesizer in accordance with the instructions attached to the synthesizer. Alternatively, it is also easy in the art to entrust the synthesis to a company or a department entrusted with the synthesis of such polynucleotides.

The nucleic acid molecule is preferably selected from exon regions of the Cx43 gene. It is also more preferable that the specificity of the sequence to the complementary region in the target gene is high. When the nucleic acid sequence is siRNA, as the region to be selected, a sequence region of AA (or CA) (N15 to 30 bases) TT containing G or C at about 50% can be exemplified as the complementary region. When the sequence described above is not found, a sequence having a terminal region of AA (N15 to 30) or CA (N15 to 30) can be used alternatively.

The gene sequence corresponding to the siRNA of human Cx43 is shown below.

| | |
|---|---|
| CAATTCTTCT TGCCGCAATT | (SEQ ID NO: 3) |

The gene sequence corresponding to the siRNA of rat Cx43 is shown below.

| | |
|---|---|
| CAATTCCTCG TGCCGCAATT | (SEQ ID NO: 4) |

The gene sequence corresponding to the siRNA of monkey Cx43 is identical to that of human Cx43.

As the nucleic acid molecule (particularly siRNA) of the present invention, the following are preferable examples.

| | |
|---|---|
| siRNA for Rats | |
| Sense: | |
| 5′-CAA UUC CUC GUG CCG CAA TT-3′ | (SEQ ID NO: 5) |
| Antisense: | |
| 5′-UUG CGG CAC GAG GAA UUG TT-3′ | (SEQ ID NO: 6) |
| siRNA for Humans and Monkeys | |
| Sense: | |
| 5′-CAA UUC UUC UUG CCG CAA TT-3′ | (SEQ ID NO: 7) |
| Antisense: | |
| 5′-UUG CGG CAA GAA GAA UUG TT-3′ | (SEQ ID NO: 8) |

In addition to the above, nucleic acid molecules having a sequence specific to Cx43 can be designed according to standard methods.

The nucleic acid molecule of the present invention can be a derivative such as a phosphorothioate derivative, which is hardly degraded by nuclease.

The nucleic acid molecule of the present invention can effectively promote the proliferation of corneal endothelial cells by being administered to the anterior chamber of the eye. As a dosage, about 1 to 50 μL, and preferably about 20 μL, of about 10 to 100 μM, and preferably about 40 μM, siRNA or antisense DNA/RNA may be administered, and sufficient effects are observed after one administration.

For the administration, it is preferable to administer by injection via the cornea. An injection needle as thin as possible is used so as not to injure the cornea. The nucleic acid molecule can be dissolved in water for injection or an appropriate buffer and administered.

The nucleic acid molecule of the present invention may be administered alone, and it is possible to enhance introduction efficiency using various reagents such as a polycation lipid liposome-based transfection reagent and a nucleic-acid introduction reagent composed of viral particles.

### EXAMPLES

The present invention will be described in more detail below based on Examples, but it goes without saying that the present invention is not limited to these Examples.

### Example 1

### Subjects and Methods

### 1) Production of Corneal Wound-Healing Model Using Rats

Wistar strain rats (male, 8 weeks of age) were used. After each rat was deeply anesthetized with pentobarbital, a needle of 30G (Nipro Medical Industries Co., Ltd.) was inserted from a limbus into an anterior chamber to take up 20 μL of anterior chamber fluid. The corneal endothelium was mildly scratched with the needle from the side of the anterior chamber to make a wound. Subsequently, in order to examine the effects of antisense oligonucleotides and RNAi for connexin 43 (Cx43) on corneal wound healing, 20 μL of 40 μM AS ODN or siRNA was injected into the anterior chamber using another needle inserted through the same incision (when the total amount of liquid in the anterior chamber is supposed to be 40 μL, the final concentration of AS ODN and siRNA in the liquid in the anterior chamber was 20 μM). After one or three days, the eye ball was removed and corneoscleral sections (diameter of 6 to 7 mm) were made.

The oligonucleotides used were as follows.

Cx43 AS ODN:

(SEQ ID NO: 9)

5′-GTA ATT GCG GCA GGA GGA ATT GTT TCT GTC-3′
Cx43 Sense ODN:

(SEQ ID NO: 10)

5′-GAC AGA AAC AAT TCC TCC TGC CGC AAT TAC-3′

The siRNA sequences used were as follows.

| | |
|---|---|
| Sense: | |
| 5′-CAA UUC CUC GUG CCG CAA TT-3′ | (SEQ ID NO: 5) |
| Antisense: | |
| 5′-UUG CGG CAC GAG GAA UUG TT-3′ | (SEQ ID NO: 6) |

2) Treatment of Non-treated Rat Corneas with Cx43 AS ODN and siRNA

Wistar strain rats (male, 8 weeks of age) were used. After each rat was deeply anesthetized with pentobarbital, a 30G needle was inserted from the limbus into the anterior chamber to take up 20 μL of anterior chamber fluid. In this case, the needle was removed without making a wound. Using another needle placed into the same incision, 20 μL of 40 μM AS ODN or RNAi was injected into the anterior chamber. After 6 and 12 hours, 1, 2 and 3 days, the eye ball was removed and corneoscleral sections were made.

### 3) Immunohistochemical Analysis

The corneoscleral sections were immersed in 1% paraformaldehyde/phosphate buffered saline (PBS) at room temperature for 5 minutes to perform fixation, and were subsequently treated with acetone at −20° C. for 10 minutes. Non-specific absorption was blocked by placing the sections in 5% skimmed milk/PBS at room temperature for 20 minutes. Treatment with 5% dextran/1% dimethylsulfoxide (DMSO)/PBS at room temperature for 10 minutes was performed three times in order to stabilize the corneoscleral structure. The corneoscleral section was trimmed using a razor into a corneal section of 4 mm×4 mm.

For immunohistochemical staining, a rabbit anti-Cx43 antibody (400 times dilution, Chemicon), a murine anti-Cx43 antibody (400 times dilution, Zymed), a rabbit anti-ZO-1 antibody (400 times dilution, Zymed) and a murine anti-Ki-67 antibody (20 times dilution, Dako) were used as primary antibodies.

The corneal sections were incubated with the anti-Ki-67 antibody for 48 hours, and incubated with the other antibodies for 24 hours at 4° C. The sections were washed three times with PBS at room temperature for 5 minutes. The corneal sections were treated at 37° C. for 90 minutes using an Alexa-488-conjugated goat anti-rabbit immunoglobulin G antibody, an Alexa-488-conjugated goat anti-mouse immunoglobulin G antibody, an Alexa-594-conjugated goat anti-rabbit immunoglobulin G antibody and an Alexa-594-conjugated goat anti-mouse immunoglobulin G antibody (all 400 times dilution, Invitrogen) as secondary antibodies. Subsequently, the sections were washed three times with PBS at room temperature for 5 minutes. In some sections, nuclei were stained by treating the sections with propidium iodine (PI, 1 mg/mL) at room temperature for 30 minutes. The corneal section was mounted on a slide glass placing the corneal endothelium side up, and embedded in Vectorshield (Vector), which is an anti-fluorescence-fading agent.

The sections were observed using a confocal laser microscope (Fluoview, Olympus) comprising an oil immersion objective lens with a magnification of 40 (Plan Apo 60, NA=1.4).

In the treatment with antisense sequences and the treatment with RNAi, each wound healing experiment was carried out in parallel with an appropriate control group, the immunohistochemical stainings were performed at the same time and the imaging using the confocal laser microscope was performed under the same conditions.

### 4) Evaluation of Wound Closure

Using the appearances of each section stained with ZO-1/PI three days after making the wound, the cell continuity was determined to evaluate wound closure.

### Results

### 1) Effects of Oligonucleotides

The corneal sections 3 days after making the wound to which the oligonucleotides were administered are shown in FIGS. 1A and 1B. These were stained with ZO-1 (green) indicating the borders of the endothelial cells and PI (red) indicating the nuclei. It has been found that the endothelial cells proliferated to completely heal the wound in the Cx43 AS ODN group (A) compared with the CX43 sense ODN group (B) in which the wound was scarcely healed.

2) Effects of siRNA

The corneal sections 3 days after making the wound to which siRNA was administered are shown in FIGS. 2A and 2B. These were stained with ZO-1 (green) indicating the borders of the endothelial cells and PI (red) indicating the nuclei. It has been found that the endothelial cells proliferated to completely heal the wound in the Cx43 siRNA group (FIG. 2A) compared with the non-functional siRNA group (FIG. 2B) in which the wound was scarcely healed.

The sequences of non-functional siRNA used were as follows.

| | |
|---|---|
| Sense: | |
| 5′-AAU UCU CCG AAC GUG UCA CGT-3′ | (SEQ ID NO: 11) |
| Antisense: | |
| 5′-GUG ACA CGU UCG GAG AAU UTT-3′ | (SEQ ID NO: 12) |

It has been further identified that siRNA has a stronger inhibitory effect on Cx43 expression and more strongly promotes the proliferation of the cells than the oligonucleotide. These results are shown in FIG. 3. The experimental conditions in FIG. 3 are shown below. In normal rats having no wound, Cx43 AS-ODN or Cx43 siRNA was administered to the anterior chamber, then corneal endothelial cells were collected over time, and immunostaining for Cx43 and Ki67 was administered thereto to obtain a Cx43-negative rate and a Ki67-positive rate. Through this experiment, it has been found that the knockdown of Cx43, not being involved in the release of contact inhibition by the wound, directly affects the proliferation of the corneal endothelial cells and enhances it. From the results in FIG. 3, it is found that treatment with siRNA is more effective than treatment with AS-ODN.

### Example 2

### Effect of hCx43-siRNA on Regeneration of Monkey Corneal Endothelium

The effect of hCx43-siRNA on the proliferation of endothelial cells was observed by administering the following hCx43-siRNA to a cultured endothelial cell sheet obtained from the cornea of a crab-eating macaque and counting the number of cells in a DNA synthesis phase.

| | |
|---|---|
| hCx43-siRNA | |
| Sense: | |
| 5′-CAA UUC UUC UUG CCG CAA TT-3′ | (SEQ ID NO: 7) |
| Antisense: | |
| 5′-UUG CGG CAA GAA GAA UUG TT-3′ | (SEQ ID NO: 8) |

As a result, the number of BrdU-positive cells was clearly increased in the hCx43-siRNA administration groups (#2-4) compared with the hCx43-siRNA non-administration group (#1), demonstrating that hCx43-siRNA (Cx43-siRNA of human and monkey have the same sequence) promotes the regeneration of monkey corneal endothelial cells.

TABLE 2

| | Administration | BrdU-positive |
|---|---|---|
| | of hCx43-siRNA | cells |
| #1 | − | 19 |
| #2 | + | 29 |
| #3 | + | 40 |
| #4 | + | 30 |

From the results of Examples 1 and 2, it has been elucidated that the nucleic acid molecule (antisense oligonucleotides, siRNA) that inhibits the expression of the human connexin 43 gene is effective for treating the a disease or disorder caused by a reduction in human corneal endothelial cells (particularly, bullous keratopathy).

### Example 3

### In Vivo Effects of hCx43-siRNA on Regeneration of Monkey Corneal Endothelium

Using a corneal endothelium disorder model from crab-eating macaques, the corneal endothelial cells of which have a poor proliferative ability in vivo similar to human beings, it is possible to evaluate the effect of administering siRNA that selectively inhibits the expression of human connexin 43 into the anterior chamber on corneal endothelium wound healing.

1. Preparation of a Connexin 43-inhibiting Drug

The following siRNA (hCx43-siRNA) was used.

| | |
|---|---|
| Sense: | 5′-CAA UUC UUC UUG CCG CAA TT-3′ |
| Antisense: | 5′-UUG CGG CAA GAA GAA UUG TT-3′ |

2. Preparation of Corneal Endothelium Disorder Models from Crab-Eating Macaques and Administration of Connexin 43-Inhibiting Drugs
1) General anesthesia: General anesthesia by intramuscular injection of ketamine hydrochloride and xylazine hydrochloride was given, and then the animal was carried from a cage to a medical table. On the medical table, inhalation anesthesia using a mask was started, then a monitor for blood pressure, electrocardiography and oxygen saturation was started and a stable general state was confirmed. Subsequently, the following surgical operation was performed.
2) Draping and disinfection: In order to prevent the infection of intraocular tissue from the skin and body hair, the face and upper body were covered with a drape for ophthalmic surgery, and disinfection with Isodine was performed.
3) Production of corneal endothelial cell disorder by percorneal cryocoagulation: The tip of a chip having a diameter of 5 mm and cooled with liquid nitrogen was adhered to the corneal center in both eyes of a crab-eating macaque, and cryocoagulation was performed for about 15 seconds until an ice ball could be identified in the anterior chamber.
4) Administration of a connexin 43-inhibiting drug: 50 μL of anterior chamber fluid was removed from the crab-eating macaque with the corneal endothelium disorder. In the right eye, 50 μL of hCx43-siRNA at a concentration of 100 μg/mL was administered to the anterior chamber. As a control, 50 μL of control siRNA at the same concentration was administered to the left eye.
5) An antibacterial drug (Tarivid [registered trade name], ophthalmic ointment) was dropped into the eye to prevent infection and complete the surgery.
6) Inhalation anesthesia was terminated, and the macaque was woken on a heat insulating mat in a cage in the recovery room. It was confirmed that the macaque awoke and there were no abnormalities, and then the macaque was returned to a cage in the breeding room.

Use of the corneal endothelium disorder model produced by such percorneal cryocoagulation is the method widely used in the study of the corneal endothelium since the 1970's. It has been reported that corneal endothelial cells in a coagulated region are caused to drop off by percorneal cryocoagulation of the corneal center, but the normal corneal endothelial cells remaining in the periphery expand and migrate, thereby resulting in wound healing and not leading to bullous keratopathy and, thus, the transparency of the cornea is recovered in about a week. In a study conducted by van Horn et al, using stump-tailed macaques, it was reported that corneal edema was observed until 24 hours after making the wound but the cornea recovered its transparency in 4 to 9 days (van Horn D L et al. Exp Eye Res, 1975). Also in a study conducted by Matsubara et al using crab-eating macaques, it was reported that the cornea was recovered in 3 days by expanding and extending the corneal endothelium after corneal cryocoagulation of a diameter of 2.5 mm (Matsubara M et al. Jpn J Opthalmology, 1982). These reports have proved that, similarly to human beings, the corneal endothelial cells in the macaque do not proliferate in vivo and that wound healing occurs through the extension and expansion of the remaining cells. Corneal endothelial cells healed in this way have a lower density than normal corneal endothelial cells, and do not recover completely even after years have passed. The same phenomenon occurs in an early phase of human bullous keratopathy. Although function is compensated for by extending and expanding the remaining cells in the early phases of such a corneal endothelium disorder, such compensation does not work over time resulting in severe bullous keratopathy.

According to a study by the present inventor, by enhancing the proliferative ability of the remaining corneal endothelial cells in such a corneal endothelium disorder during the early phases, it becomes possible to heal the wound with corneal endothelial cells that have a higher density and are morphologically healthy.

When a corneal endothelial cell disorder is created in vivo in a crab-eating macaque, the cornea is covered with endothelial cells derived from the expansion and extension of the remaining corneal endothelial cells in the periphery about one week after transplantation, and recovers its transparency to some extent. The effect of the administered drug on corneal endothelial cell recovery can be evaluated by comparing the right and left eyes for the morphology and density of the corneal endothelial cells and corneal thickness, which is an indicator of corneal endothelial cell function when the cornea recovers its transparency. That is, when a proliferation promotion effect on the corneal endothelial cells is observed by administering the drug, the density of the corneal endothelial cells becomes higher compared with that of the control eye, and the corneal thickness becomes thinner because the pump function of the corneal endothelial cells is good.

In the corneal endothelium disorder model produced by percorneal cryocoagulation, corneal edema peaks in strength from immediately after treatment to the following day. In the early phases after the operation, the general states, the appetites and behavior of the macaques were observed three times a day. When it was determined that feeding and water intake were insufficient, the appropriate treatment e.g., oral supply (Enrich [registered trade name]) or drop infusion of nutrition and water were rapidly performed.

### 3. Observation of Anterior Ocular Segment

1) Observation of general state: Appetite, excretion, fur coat and behavior were observed and it was confirmed that there was no problem in their general state.
2) Observation of anterior ocular segment by slit lamp (slit lamp microscope)

The anterior ocular segment was observed before the surgical procedure, on the day after the procedure, 3 days, 7 days and 1, 3 and 4 weeks after the procedure using a slit lamp, a corneal thickness measurement apparatus and a corneal endothelium specular observation apparatus. The slit lamp is a biological microscope widely used in ophthalmic practice, and detailed observation and photography can be performed by placing the face on a chin support and throwing a thin light. The time period required for the above observation is about 10 minutes, and because it is a non-invasive examination, there is no pain and distress, but it is necessary to stand still for several tens of seconds. Thus, general anesthesia was administered by intramuscular administration of ketamine hydrochloride and xylazine hydrochloride.

### 4. Histological Study

When wound healing was completed, the crab-eating macaque was euthanized and the corneal tissues were removed. The corneal endothelial cells were subjected to histological study using immunohistochemical techniques.

Through the above experiments, it is confirmed that siRNA (hCx43-siRNA) of the present invention is effective in the corneal endothelium disorder model produced by percorneal cryocoagulation of crab-easting macaques, and it is demonstrated that siRNA of the present invention also promotes the proliferation of the corneal endothelial cells in a human being whose corneal endothelial cells have poor proliferative ability, similarly to the macaque, and is effective for the treatment of a disease or disorder such as bullous keratopathy caused by a reduction in corneal endothelial cells.

### INDUSTRIAL APPLICABILITY

The present invention can promote the proliferation of human corneal endothelial cells by inhibiting the expression of the connexin 43 gene, and is effective for the prevention or treatment of bullous keratopathy.

Also according to the present invention, corneal endothelial cells reduced due to intraocular surgery or laser therapy can be recovered, and, for example, the present invention can make it easier to perform cataract treatments on the elderly.

### BRIEF DESCRIPTION OF DRAWINGS

FIG. 1 is a view showing the results of treatment with antisense oligonucleotides: (A) Cx43 AS ODN treatment group and (B) Cx43 sense ODN treatment group;

FIG. 2 is a view showing the results of treatment with siRNA: (A) Cx43 siRNA treatment group and (B) non-functional siRNA treatment group (control); and

FIG. 3 is a view showing the results of the comparison of treatment with antisense oligodeoxynucleotide (ODN) and treatment with siRNA through the rates of Cx43-negative cells [Cx43(−)] and Ki67-positive cells [Ki67(+)]: (A) AS ODN treatment and (B) siRNA treatment.

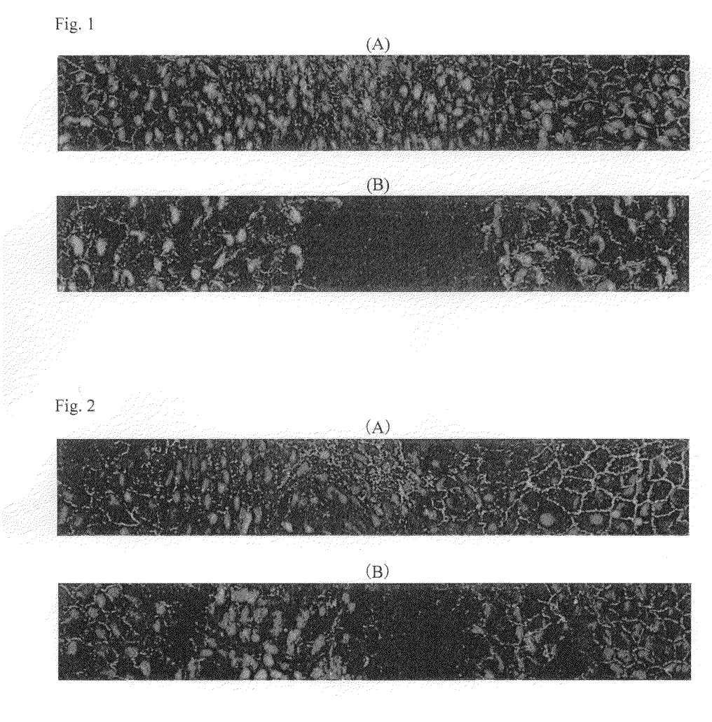

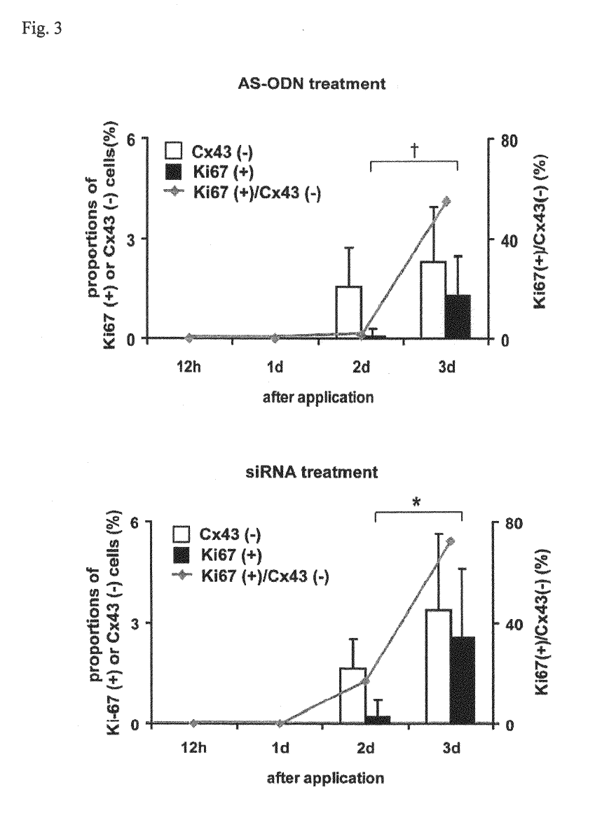

Sequence Listing

1. A treatment agent of bullous keratopathy comprising as an active component siRNA containing a polynucleotide sequence of 19 to 21 consecutive nucleotides having a sequence complementary to a connexin 43 gene.

2. (canceled)

3. The treatment agent according to claim 1 which is in a form of a formulation for administering to an anterior chamber of an eye.

4. (canceled)

5. The treatment agent according to claim 1 wherein said siRNA is siRNA having the following sequences:

| | |
|---|---|
| sense: | 5′-CAA UUC UUC UUG CCG CAA TT-3′ |
| and | |
| antisense: | 5′-UUG CGG CAA GAA GAA UUG TT-3′ |

for human connexin 43.

6. Use of siRNA containing a polynucleotide sequence of 19 to 21 consecutive nucleotides having a sequence complementary to a connexin 43 gene, for treating bullous keratopathy.

7. (canceled)

8. The use according to claim 6 for administering said siRNA to an anterior chamber of an eye.

9. (canceled)

10. The use according to claim 6 wherein said siRNA is siRNA having the following sequences:

| | |
|---|---|
| sense: | 5′-CAA UUC UUC UUG CCG CAA TT-3′ |
| and | |
| antisense: | 5′-UUG CGG CAA GAA GAA UUG TT-3′ |

for human connexin 43.

11. A method for treating bullous keratopahy, wherein siRNA containing a polynucleotide sequence of 19 to 21 consecutive nucleotides having a sequence complementary to a connexin 43 gene is administered to a patient having bullous keratopathy.

12. (canceled)

13. The method according to claim 11 wherein said siRNA is administered to an anterior chamber of an eye.

14. (canceled)

15. The method according to claim 11 wherein said siRNA is siRNA having the following sequences:

| | |
|---|---|
| sense: | 5′-CAA UUC UUC UUG CCG CAA TT-3′ |
| and | |
| antisense: | 5′-UUG CGG CAA GAA GAA UUG TT-3′ |

for human connexin 43.

16. A pharmaceutical composition for treating bullous keratopathy, comprising an effective amount of siRNA containing a polynucleotide sequence of 19 to 21 consecutive nucleotides having a sequence complementary to a connexin 43 gene, and a pharmaceutically acceptable carrier, excipient or diluent.

17. (canceled)

18. The pharmaceutical composition according to claim 16 which is in a form of a formulation for administering to an anterior chamber of an eye.

19. (canceled)

20. The pharmaceutical composition according to claim 16 wherein said siRNA is siRNA having the following sequences:

| | |
|---|---|
| sense: | 5′-CAA UUC UUC UUG CCG CAA TT-3′ |
| and | |
| antisense: | 5′-UUG CGG CAA GAA GAA UUG TT-3′ |

for human connexin 43.

21. (canceled)

22. (canceled)

23. (canceled)

24. (canceled)

25. (canceled)

26. A treatment method for allowing an intraocular surgery, wherein before the intraocular surgery, siRNA containing a polynucleotide sequence of 19 to 21 consecutive nucleotides having a sequence complementary to a connexin 43 gene is administered to a patient wherein corneal endothelial cells are reduced, and bullous keratopathy is likely to occur due to the surgery but the intraocular surgery is required.

27. (canceled)

28. The method according to claim 26 wherein said siRNA is administered to an anterior chamber of an eye.

29. (canceled)

30. The method according to claim 26 wherein said siRNA is siRNA having the following sequences:

| | |
|---|---|
| sense: | 5′-CAA UUC UUC UUG CCG CAA TT-3′ |
| and | |
| antisense: | 5′-UUG CGG CAA GAA GAA UUG TT-3′ |

for human connexin 43.

31. siRNA having the following sequences:

| | |
|---|---|
| sense: | 5′-CAA UUC UUC UUG CCG CAA TT-3′ |
| and | |
| antisense: | 5′-UUG CGG CAA GAA GAA UUG TT-3′ |

for human connexin 43.
